(19) United States
(12) Patent Application Publication
(10) Pub. No.: US 2014/0005605 A1
(43) Pub. Date: Jan. 2, 2014

(54) USE OF QUORUM SENSING INHIBITORS AND BIOFILM DISPERSING AGENTS FOR CONTROLLING BIOFILM-ASSOCIATED IMPLANTABLE MEDICAL DEVICE RELATED INFECTIONS

(71) Applicants: Richard Samade, Northridge, CA (US); Prashant Dinesh, Bangalore (IN); Yelena Nabutovsky, Sunnyvale, CA (US); Gene A. Bornzin, Simi Valley, CA (US); John W. Poore, South Pasadena, CA (US); Annapurna Karicherla, Valencia, CA (US); Nirav Dalal, Porter Ranch, CA (US)

(72) Inventors: Richard Samade, Northridge, CA (US); Prashant Dinesh, Bangalore (IN); Yelena Nabutovsky, Sunnyvale, CA (US); Gene A. Bornzin, Simi Valley, CA (US); John W. Poore, South Pasadena, CA (US); Annapurna Karicherla, Valencia, CA (US); Nirav Dalal, Porter Ranch, CA (US)

(73) Assignee: PACESETTER, INC., Sylmar, CA (US)

(21) Appl. No.: 13/536,848

(22) Filed: Jun. 28, 2012

(51) Int. Cl. C09D 133/10; C09D 129/04; C09D 167/02; C09D 139/06; C09D 175/04; A61M 5/00; C09D 167/00

(52) U.S. Cl. CPC C09D 133/10; A61M 5/00; C09D 129/04; C09D 167/00; C09D 139/06; C09D 175/04; C09D 167/02
USPC 604/131; 524/558; 524/557; 524/599; 524/548; 524/590; 524/606

(57) ABSTRACT

A coating on at least a portion of an implantable medical device includes a polymer and an agent that inhibits the formation of biofilms. The agent inhibiting the formation of a biofilm includes a quorum sensing inhibitor (QSI), a biofilm dispersing agent (BDA) or both. The agent may also be delivered via an actuator associated with the implantable medical device.

### BACKGROUND OF THE INVENTION

1. Field of the Invention

The present invention relates generally to implantable medical devices and, more specifically, to using quorum sensing inhibitors and/or biofilm dispersing agents to control biofilm-associated implantable medical device related infections.

2. Background Art

Implantable device-related infections (DRIs) are a serious problem that arises in about 2% of de novo implants of cardiac rhythm management (CRM) devices, such as cardiac resynchronization therapy devices (CRTs), implanted cardioverter-defibrillators (ICDs), and permanently-implanted pacemakers (PPMs). The incidence is higher for patients that are diabetic, on kidney dialysis, receiving device replacements, or undergoing lead revisions. In addition, recent studies suggest that DRIs are increasing due to growth in device complexity and are more prevalent when implanting physicians are less experienced. A DRI is extremely costly—combined medical and surgical treatment of a DRI is approximately $25,000 for a PPM and $50,000 for an ICD—and causes the patient to be susceptible to potentially fatal complications.

The time course of infection development is not fully understood and varies greatly. However, it has been reported that approximately thirty percent of infections arise less than one month post-implant, another thirty-five percent occur between one month and twelve months post-implant, and the remainder appear more than a year post-implant See Lekkerkerker et al., “Risk factors and time delay associated with cardiac device infections: Leiden device registry,” Heart 95:715-720 (2009).

There is a dearth of technologies available to prevent DRIs. One known product that is commerically available is the AIGIS_{RX} Anti-Bacterial Envelope, manufactured by TyRx Pharma, Inc. of Monmouth Junction, N.J., USA. This anti-microbial pouch, designed for use with PPMs and ICDs, is a polypropylene mesh that is shaped into a pocket and is impregnated with antibiotics. The PPM or ICD can is placed into the envelope and the covered device is subsequently implanted. The antibiotics (minocycline and rifampin) are eluted over a minimum period of seven days in order to prevent DRI. There are several shortcomings to this approach—it requires the implanting physician to execute an extra step of placing the device in the pocket, it adds volume to the device can (which increases patient discomfort post-surgery), device replacement or explant is more difficult (due to growth of tissue into the mesh), and it acts only for a short period of time (therefore not addressing long-term DRIs). A previous in vivo study of AIGIS_{RX} demonstrated that it was able to prevent the formation of biofilms composed of Acinetobacter baumannii bacteria. However, the efficacy of the AIGIS_{RX} against biofilm formation was demonstrated only seven days postsurgery and could not be expected to prevent biofilms that appeared at later time periods (when the antibiotic combination used falls below the minimum inhibitory concentration).

Several studies have acknowledged that conventional treatments, such as antibiotics, become significantly less efficacious against potential bacteria infections once they transition from a planktonic mode to a biofilm-associated sessile mode. A more desirable solution would be to either prevent biofilms from forming or to dissolve the biofilm (thereby returning their constituents into a planktonic mode). Once either of those actions is accomplished, bacteria will become more susceptible to destruction by antimicrobial therapies or the innate and adaptive components of a patient's immune system.

Bacteria are known to use certain molecules, known as autoinducers, for a system of interspecies and intraspecies communication, termed “quorum sensing.” Autoinducers that bind to receptors on a target bacterium can initiate a plethora of changes in gene expression and subsequent phenotypes conducive to biofilm formation, such as extracellular matrix production and bacterial attachment. Recent research activities have been examining methods for interfering with the bacterial quorum sensing system and thereby preventing biofilm formation. One common bacterial species associated with DRIs, Pseudomonas aeruginosa, was found to have inhibited biofilm formation in an in vivo study when mice were treated with the quorum sensing inhibitor (QSI) furanone C-30, which promoted clearance by the murine immune system. Another bacteria frequently associated with DRIs, Staphylococcus aureus, was discovered to have significantly decreased populations in an in vivo rat model when a known QSI for Staphylococcus aureus, RNA III Inhibiting Peptide (RIP), was used in combination with antibiotics (compared to antibiotic use alone). Currently, commercial entities such as Biosignal, Ltd. (Eveleigh, Australia) and Quiescence Technologies, LLC (Melbourne, Fla., U.S.A.) are evaluating potential clinical uses of QSIs.

The other alternative to a prophylactic disruption of the Quorum Sensing System (QSS) by QSIs and subsequent prevention of biofilm formation, is to disperse bacteria in an already-present biofilm, which is also the subject of several investigations. One such example of biofilm dispersal agents (BDAs) is alginate lyase, an enzyme that degrades the alginate extracellular matrix formed by Pseudomonas aeruginosa and was found to increase the in vivo effectiveness of an antibiotic in a rabbit model of infective endocarditis. Another BDA is the dispersin B polypeptide, which was demonstrated to reduce the rate of Staphylococcus aureus colonization of catheters in an in vivo study with a rabbit model of subcutaneous infection.

### BRIEF SUMMARY OF THE INVENTION

The invention relates to the use of QSIs, BDAs, or a combination thereof, as a method for controlling DRIs associated with biofilms in implantable medical devices (“IMDs”). These treatments are intended to provide protection for IMDs from biofilm-associated DRIs under both short-term (e.g., 0-28 days) and long-term (e.g., greater than 28 days) scenarios. The QSIs or BDAs can be either allowed to elute for a steady period of time or have active controls that release these therapeutic agents in response to sensors on the IMD or external commands (such as by a physician).

The rate and amount of elution can be controlled by active means, controlling the amount of QSI or BDA coating applied on the CRM device, or by using various numbers and sizes of crystalline forms of QSIs or BDAs.

This approach has several advantages including, for example:
(a) The inclusion of active means of QSI or BDA release or use of crystals of QSIs or BDAs permits a longer elution time (and hence a longer period of biofilm prevention and control) than would be possible with simply a coating on the CRM device;
(b) QSIs or BDAs should not have any toxic effects on mammalian cells;
(c) QSIs or BDAs may be used that are specifically tailored to their bacterial targets;
(d) QSIs or BDAs will discourage bacteria from assuming pro-biofilm phenotypes, increasing the susceptibility of now-planktonic bacteria to destruction by antimicrobial therapies or the host immune system;
(e) Use of QSIs or BDAs in combination with antibiotics may render oral antibiotics more effective against infection. In this scenario, one would not need to rely heavily on antibiotic coatings for protection against DRIs. Furthermore, a physician would be able to tailor, for example, an antibiotic cocktail for the specific bacterial species causing a patient's infection; and
(f) Use of QSIs or BDAs does not lead to resistance to these agents.

The primary purpose of using either QSIs, BDAs, or both, as methodologies for controlling DRIs is to either preclude the emergence of bacterial biofilms (in the case of QSIs) or to dismantle pre-existing biofilms (in the case of BDAs) that could form on IMD cans, headers, or leads. Once bacteria are rendered incapable of aggregating together in biofilms, they are restricted to a free-floating planktonic form. In this state, bacteria are more susceptible to elements of the patient's innate and adaptive immune system (such as complement, antibodies, and phagocytes), as well as conventional (such as antibiotics) and novel antimicrobial treatments.

Since many QSIs and BDAs are specific to certain bacterial species, the QSIs and BDAs chosen for controlling DRIs in CRM devices should be specially formulated against bacterial species commonly found infecting these devices, such as Staphylococcus aureus and Pseudomonas aeruginosa. As an example, a QSI and BDA combination effective against Staphylococcus aureus would use RNA III Inhibiting Peptide (RIP) (as the QSI) and dispersin B polypeptide (as the BDA). The different receptors and signal transduction pathways of Staphylococcus aureus would not permit this formulation to be as effective against Pseudomonas aeruginosa, which would require, for example, a combination of furanone C-30 (the QSI) and alginate lygase (the BDA). However, research studies show some promise for certain QSIs and BDAs to have broad spectrum action against several bacterial species. As an example, the QSI 4-nitro-pyridine-N-oxide and the BDA cis-2-decanoic acid have been reported to be effective against biofilms formed by both Staphylococcus aureus and Pseudomonas aeruginosa.

In addition to the above-described methodologies, other possible tailored formulations of the device can impart either a priori determined elution profiles of these compounds or elution rates that are dynamically controlled in response to DRIs. Methods for achieving the former include application of a coating on the CRM device or encasing the device in a polymer mesh. The portion of the coatings that do not include the QSIs or BDAs can further be classified into one of two subcategories: hydrophilic coatings or hydrophobic coatings. Hydrophilic coatings could be applied to the device by either chemical immersion or vapor deposition methods. These coatings could then be coated with the desired QSIs, BDAs, or both as a uniform layer (via a second chemical immersion method). Hydrophobic coatings, such as polyurethane or Dacron, offer the alternative possibility to fashion an alloyed material composed of the parent hydrophobic material and primarily hydrophilic QSI or BDA compounds, in a manner similar to that done for polyurethane-antibiotic alloys. This process, using a solvent casting technique, would yield crystalline precipitates of the QSI or BDA compounds in the alloy (since the parent material and QSI or BDA compounds repel each other in order to minimize entropy). These crystals could be modified in size and number such that a multitude of possible elution profiles could be obtained.

Polymer meshes, which could be composed of polyethylene glycol, polypropylene, or another polymer type, could be used to bind to the QSIs or BDAs and control the rate of elution. These polymer meshes would encase the CRM device and may be applied either during the manufacturing process by sliding it on the device or during the implant process.

In an alternate embodiment, dynamic control of QSI and/or BDA release could be achieved using one or more actuators to secrete an agent from the CRM device. The control signal(s) used to activate these actuators could be delivered externally by a physician using an external programmer, in response to an infection diagnosis in a patient with a positive bacterial culture. Alternatively, the release signal(s) may occur automatically, on a pre-set schedule or in response to the detection of bacteria by sensors on the CRM device. The release may be a constant elution of QSIs, BDAs, or both for long-term protection. The emergence of an acute infection, by contrast, could trigger a large bolus-like release of QSIs and/or BDAs in order to obtain immediate anti-biofilm therapy. An additional advantage of actuator-delivered agents is the ability to control the rate and time-profile of QSI and BDA elution, in response to various inputs from the external programmer or the implanted bacterial sensors. Depending upon the species of bacteria detected (e.g. by either the implanted bacterial sensor or laboratory cultures), the actuators could release only a portion of their QSI and/or BDA stores that are targeted specifically against the bacteria species present (which may be in either a planktonic or biofilm state).

### BRIEF DESCRIPTION OF THE DRAWINGS/FIGURES

The accompanying drawing, which is incorporated herein and forms part of the specification, illustrates the apparatus presented herein. Together with the detailed description, the drawing further serves to explain the principles of, and to enable a person skilled in the relevant art(s) to make and use, the apparatus and methods presented herein.

FIG. 1 is a simplified diagram illustrating an exemplary implantable cardiac therapy device (ICTD) in electrical communication with a patient's heart by means of leads suitable for delivering multi-chamber stimulation and pacing therapy, and for detecting cardiac electrical activity.

### DETAILED DESCRIPTION OF THE INVENTION

For purposes of illustration, the present invention is described in the environment of an implantable cardiac therapy device (ICTD). The invention, however, has application to any implantable medical device (“IMD”). As used herein, the term implantable medical device or IMD includes ICTDs (e.g., cardiac rhythm management devices, cardiac resynchronization therapy devices, cardioverter-defibrillators, and pacemakers), implantable infusion pumps, implantable wireless sensors, and the like.

An ICTD is a physiologic measuring device and therapeutic device that is implanted in a patient to monitor cardiac function and to deliver appropriate electrical therapy, for example, pacing pulses, cardioverting and defibrillator pulses, and drug therapy, as required.

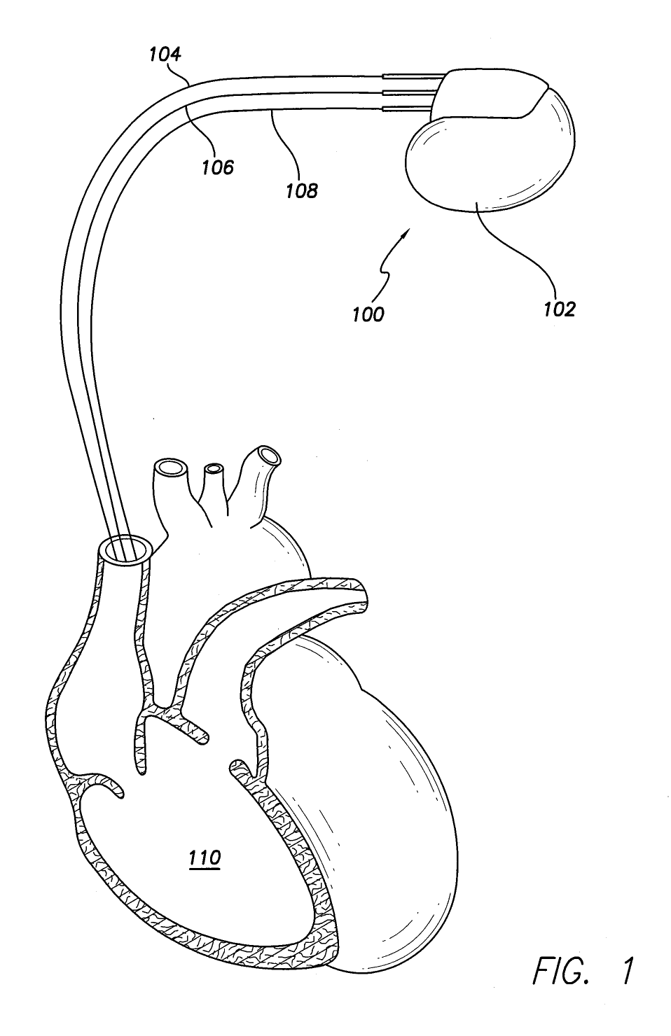

FIG. 1 shows an exemplary ICTD 100 in electrical communication with a patient's heart 110 by way of three leads 104, 106, 108. ICTD 100 includes a housing 102 that is often referred to as the “can”, “case” or “case electrode.” Leads 104, 106 and 108 are suitable for delivering multi-chamber stimulation and shock therapy to heart 110 as is known in the art. For example, ICTD 100 and leads 104, 106, 108 may be of the type described in U.S. Pat. No. 8,005,533 to Farazi, which is incorporated herein by reference as if reproduced in full below.

In various embodiments, housing 102 of ICDT 100 may be formed from any biocompatible material having appropriate mechanical and electrical properties for the particular application. For example, suitable materials include titanium, titanium alloys (such as nickel titanium), nickel alloys, stainless steel, stainless steel alloys, platinum, platinum alloys and mixtures thereof. In other embodiments, suitable materials may include polymeric materials such as polyurethanes, polyamides, polyetheretherketones (PEEK), polyether block amides (PEBA), polytetrafluoroethylene (PTFE), silicones, and mixtures thereof. In yet other embodiments, suitable materials may include, for example, ceramic materials such as zirconium ceramics and aluminum-based ceramics.

According to an embodiment, at least a portion of housing (i.e., can) 102 of ICDT 100 includes a coating that inhibits the formation of biofilms. The coating comprises a polymer and an agent that inhibits the formation of biofilms. In a one embodiment, the polymer comprises a hydrophilic polymer. Exemplary hydrophilic polymers include poly(hydroxyethyl methacrylate) (PHEMA), poly(vinyl) alcohol (PVA), poly(carboxylic acids), and poly(N-vinyl-2-pyrollidone) (PNVP). In certain embodiments, the hydrophilic polymer is applied using chemical immersion techniques or vapor deposition techniques on to housing 102.

In another embodiment, the polymer comprises a hydrophobic polymer. Exemplary hydrophobic polymers include, but are not limited to, polyurethane and Dacron. In certain embodiments, the hydrophobic coatings are applied on to housing 102 using a solvent casting technique.

In one embodiment, the agent is a quorum sensing inhibitor (QSI). Exemplary QSIs include, but are not limited to, 4-nitropyridine-N-oxide, furanone C-30 and RNA III inhibiting peptide. In one embodiment, the QSI is covalently bonded to the polymer coating before or after application of the polymer on to the housing. In another embodiment, the QSI is non-covalently dispersed in the polymer, either before or after application of the polymer on to the housing.

In another embodiment, the agent is a biofilm dispersal agent (BDA). Exemplary BDAs include, but are not limited to, cis-2-decanoic acid, alginate lyase and dispersin B polypeptide. In one embodiment, the BDA is covalently bonded to the polymer coating before or after application of the hydrophilic polymer on to the housing. In another embodiment, the BDA is non-covalently dispersed in the polymer, either before or after application of the polymer on to the housing.

In a further embodiment, the polymer coating comprises both a QSI and a BDA in amounts effective to prevent the formation of or disrupt a biofilm on said housing.

In a further embodiment, the QSI or BDA or both is present in an amount effective to prevent the formation of a biofilm on said housing. In certain embodiments the QSI or BDA or both is present in a crystalline or amorphous form in the polymer coating.

In certain embodiments, the polymer coating is an alloyed material composed of the parent hydrophobic material and primarily hydrophilic QSI or BDA compounds. The QSIs or BDAs form crystalline precipitates in the alloy (since the parent material and QSI or BDA compounds repel each other in order to minimize entropy). In certain embodiments, the crystals size and number are modified such that a multitude of possible elution profiles are obtained.

In some embodiments the QSI or BDA may be selected based on their selectivity for bacteria that are expected to form a biofilm on ICTD 100.

Another embodiment of the invention is a method for manufacturing an ICTD comprising the steps of:
forming a housing for the ICTD from a biocompatible material; and
applying to an exterior surface of the housing a coating selected from the group consisting of a hydrophilic polymer and a hydrophobic polymer, wherein the coating further comprises an agent that inhibits the formation of biofilms.

Accordingly, an exemplary ICTD may be partially or completely coated with a hydrophilic polymer using a chemical immersion or vapor deposition method, and a QSI or a BDA, or both, applied on to the hydrophilic coating via a second chemical immersion layer.

Another exemplary ICTD may be partially or completely coated with a hydrophobic polymer such as Dacron, applied using a solvent casting technique. A QSI or a BDA, or both, is applied along with the hydrophobic coating. The QSI or BDA forms crystals, which slowly elute the active compound, thereby either preventing the formation of a biofilm or disrupting the formed biofilm.

In an alternate embodiment, rather than coating the ICTD with the agent, an actuator such as an electrochemical or electromechanical actuator (e.g., an infusion pump) can be used to control the release of the QSA or BDA. In one example embodiment, an infusion pump may be integrated into housing 102 and one or a plurality of ports (i.e., openings) may be formed in housing 102 to provide a path for an agent to be infused from a reservoir within housing 102 to the exterior of housing 102. For example, if implemented in an ICTD such as that disclosed by the above-referenced U.S. Pat. No. 8,005,533 an infusion pump may be implemented in the ICTD housing under control of the ICTD's controller.

A physician may provide control signals to the controller via the external programmer. For example, a physician using an external programmer could, in response to an infection diagnosis in a patient with a positive bacterial culture, provide control signals commanding the infusion pump to deliver an agent. Alternatively, the release signal may be programmed into the controller to occur automatically, either on a set schedule or in response to the detection of bacteria by sensors associated with the ICDT. In either case, the release may be a constant infusion of QSIs, BDAs, or both for long-term protection or, in the case of an acute infection, the release could be a large bolus-like release of QSIs and/or BDAs in order to obtain immediate anti-biofilm therapy.

An advantage of using such actuators is the ability to carefully control, at different rates and times, which QSIs and BDAs are released in response to various inputs from the external programmer or the internal biosensor. Depending upon the species of bacteria detected (e.g, by either the biosensor or laboratory cultures), the actuator (or actuators) could release only a portion of their QSI and/or BDA stores that are targeted specifically against the bacteria species present (which could form or already have formed a biofilm).

Example implantable drug infusion pumps are disclosed in U.S. Pat. No. 7,831,310 to Lebel et al., U.S. Pat. No. 5,328,460 to Lord et al., U.S. Pat. No. 4,573,994 to Fischell et al., and U.S. Pat. No. 4,373,527 to Fischell et al., each of which is incorporated herein by reference as if reproduced in full below. Based on the teachings set forth herein a person skilled in the relevant arts would understand how to modify the ICDT (such as that disclosed in the above-referenced U.S. Pat. No. 8,005,533) and to adapt an infusion pump (such as one disclosed in one of U.S. Pat. Nos. 7,831,310; 5,328,460; 4,573,994; or 4,373,527) for inclusion in the ICDT.

As an alternative to including an infusion pump in the housing 102 of ICDT 100, an infusion pump may be co-located at the implantation site with ICDT 100 and may be controlled to deliver an agent to the exterior surface of ICDT 100. In yet another embodiment, the infusion pump may be implanted at a site remote from ICDT 100, and a catheter may be used to convey to the site of ICDT 100 an agent delivered by the infusion pump.

Yet another embodiment of the aforementioned system for preventing or dissolving biofilms uses a store of antibiotics, retained in actuators that may be programmed to release in response to either a timed schedule or from a release signal. These antibiotics would be released in conjunction with QSIs or BDAs so that bacteria are prevented from assuming a biofilm phenotype and are killed as well. Antibiotics could be selected so that they have broad-spectrum coverage (i.e. they address infections due to Gram-positive organisms, such as Staphylococcus aureus, and Gram-negative organisms, such as Pseudomonas aeruginosa). Alternatively, an array of actuators and associated wells containing different antibiotics could be fashioned so that a combination tailored for a particular bacterial species could be employed. This customization of the antibiotic delivery would depend on either implanted bacterial sensors that identifies the pathogen present or an input signal from an external programmer, which could be commanded by a physician to deliver a certain combination on the basis of cultures obtained from the patient. In this manner, antibiotics can be released from the anti-microbial device (in conjunction with QSI or BDA administration) with a steady elution profile so that long-term anti-bacterial protection is achieved or it can be released as a bolus in order to address acute infections.

Designs of the antimicrobial device that use actuators could be fashioned such that the wells containing either QSIs, BDAs, or possibly antibiotics would attach to a network of internal tubing that permits replenishment of those chemical stores in the device. A minimally invasive procedure to refill the wells could entail insertion of a syringe configured with internal tubing corresponding to each QSI, BDA, or antibiotic hypodermically. The syringe would contact the associated port in the antimicrobial device and, after locking with the device, could begin to refill the chemical wells. The syringe would be of small French size and would entail a wound created during the process on the order of that created by an indwelling catheter, but only for the brief period of time necessary to replenish QSI, BDA, and antibiotic stocks in the antimicrobial device.

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

What is claimed is:

1. An implantable medical device comprising a coating on at least a portion of the device, wherein the coating comprises a polymer and an agent that inhibits the formation of biofilms.

2. The implantable medical device of claim 1, wherein the polymer comprises a hydrophilic polymer.

3. The implantable medical device of claim 2, wherein the agent comprises a quorum sensing inhibitor.

4. The implantable medical device of claim 3, wherein the quorum sensing inhibitor is selected from the group consisting of 4-nitropyridine-N-oxide, furanone C-30 and RNA III inhibiting peptide.

5. The implantable medical device of claim 3, wherein the quorum sensing inhibitor is covalently bonded to the hydrophilic polymer.

6. The implantable medical device of claim 2, wherein the agent comprises a biofilm dispersal agent.

7. The implantable medical device of claim 6, wherein the biofilm dispersal agent is selected from the group consisting of cis-2-decanoic acid, alginate lyase and dispersin B polypeptide.

8. The implantable medical device of claim 6, wherein the biofilm dispersal agent is covalently bonded to the hydrophilic polymer.

9. The implantable medical device of claim 1, wherein the polymer comprises a hydrophobic polymer.

10. The implantable medical device of claim 9, wherein the agent comprises a quorum sensing inhibitor.

11. The implantable medical device of claim 10, wherein the quorum sensing inhibitor is selected from the group consisting of 4-nitropyridine-N-oxide, furanone C-30 and RNA Ill inhibiting peptide.

12. The implantable medical device of claim 10, wherein the quorum sensing inhibitor is covalently bonded to the hydrophobic polymer.

13. The implantable medical device of claim 9, wherein the agent comprises a biofilm dispersal agent.

14. The implantable medical device of claim 13, wherein the biofilm dispersal agent is selected from the group consisting of cis-2-decanoic acid, alginate lyase and dispersin B polypeptide.

15. The implantable medical device of claim 13, wherein the biofilm dispersal agent is covalently bonded to the hydrophobic polymer.

16. The implantable medical device of claim 2, wherein the agent is dispersed within the hydrophilic coating.

17. The implantable medical device of claim 9, wherein the agent is dispersed within the hydrophobic coating.

18. A method of controlling the formation of a biofilm on an implantable medical device comprising applying a coating on a portion of said device, wherein the coating is selected from the group consisting of a hydrophilic polymer and a hydrophobic polymer, and wherein the coating further comprises an agent that inhibits the formation of biofilms.

19. The method according to claim 18, wherein the agent is selected from the group consisting of a quorum sensing inhibitor and a biofilm dispersal agent.

20. A method for manufacturing an implantable medical device comprising: forming a housing for the implantable medical device from a biocompatible material; and
applying to an exterior surface of the housing a coating selected from the group consisting of a hydrophilic polymer and a hydrophobic polymer, wherein the coating further comprises an agent that inhibits the formation of biofilms.

21. The method of claim 20, wherein the agent is selected from the group consisting of a quorum sensing inhibitor and a biofilm dispersal agent.

22. An implantable medical device comprising:
a housing; and
an actuator disposed within the housing, wherein the actuator is configured to deliver to an exterior surface of the housing an agent that inhibits the formation of biofilms, wherein the agent is selected from the group consisting of a quorum sensing inhibitor and a biofilm dispersal agent.

23. The implantable medical device of claim 22, wherein the quorum sensing inhibitor is selected from the group consisting of 4-nitropyridine-N-oxide, furanone C-30 and RNA III inhibiting peptide.

24. The implantable medical device of claim 22, wherein the biofilm dispersal agent is selected from the group consisting of cis-2-decanoic acid, alginate lyase and dispersin B polypeptide.
